(19) United States
(12) Patent Application Publication
(10) Pub. No.: US 2023/0184637 A1
(43) Pub. Date: Jun. 15, 2023

(54) AN APPARATUS FOR PROCESSING BIOLOGICAL MATERIAL

(71) Applicant: Andrea RAGGI, Serravalle (SM) (SM)

(72) Inventor: Andrea RAGGI, Serravalle (SM) (SM)

(21) Appl. No.: 17/924,279

(22) PCT Filed: May 13, 2021
(86) PCT No.: PCT/IB2021/054097

§ 371 Date: Nov. 9, 2022

(30) Foreign Application Priority Data
May 13, 2020 (IT) 102020000010879

(51) Int. Cl. G01N 1/28; B01L 3/00

(52) U.S. Cl. CPC G01N 1/286; B01L 3/502; G01N 2001/2873; B01L 2300/049; B01L 2300/027; B01L 2300/043; B01L 2400/02; B01L 2300/08; B01L 2400/0496

(57) ABSTRACT

An apparatus for processing biological material includes a supporting structure, provided with a seat and with a rotation device supplied with constraining means, and a container for biological material, provided with a shaped body, with a base, shaped to match the seat of the supporting structure, with at least one valve (24a) for extracting the biological material, and with movable cutting means equipped with coupling means for coupling to the constraining means of the rotation device, in such a way as to acquire a relative motion relative to the container.

### TECHNICAL FIELD

The present invention concerns a container and an apparatus for processing biological material, and the related method for processing biological material.

Over the last decades, the interest in regenerative medicine has favoured the increase of studies in the field of stem cell biology; among these, adult stem cells, Mesenchymal Stem Cells or MSCs, present a number of features which allowed their wide use in regenerative medicine. They are in fact easily available in various human tissue areas, have a considerable clonogenic ability “in vitro” and a high osteogenic, chondrogenic and adipogenic cellular differentiation potential, as well as a considerable immunoregolatory, anti-inflammatory and regenerative function.

Among stem cells resulting from different anatomical areas, it has been documented that those isolated from adipose tissue have the ability to differentiate into many different cell types and proliferate relatively easily.

The adipose tissue is a type of loose connective tissue, from which many molecules can be extracted, like for example FGF-2, VEGF, EGF, TGFβ1, ANG-1, IGF-1, IL-1RA, IL-6, that assure the growth of fibroblasts, adipocytes and stromal elements responsible for the formation of tissues, acting jointly with a positive effect also on inflammatory phenomena.

Being particularly rich in mesenchymal cells, which are suitable for differentiation and rich in growth factors, in the last few years the adipose tissue has been used in the treatment of patients suffering from chronic pathologies not responding to conventional treatments, and not only.

### BACKGROUND ART

Most of the known methods for processing biological material, in particular human, intended for the selection of stem cells and growth factors, are based on immunolabeling and gene selection techniques.

Unfortunately, these techniques have several drawbacks which do not ensure their effectiveness as expected: in fact, the first are associated with the risk of cell damage or cell incompatibility with clinical use, the second entail high costs, long time for implementation and the use of highly qualified personnel.

The tools traditionally used to process this kind of biological material make use of equipment dedicated to the mechanical breaking up of the material procured, generally, from the same patient, and for this reason called “autologous”.

Some systems are known, recognisable from their associated trademarks, like for example, Puregraft®, Celution®, Icellator®, which are mainly based on devices for the filtration and centrifugation of the obtained material, with proper addition of enzymes; however, none of these methods allow to obtain a particular abundance of regenerative factors and the several transfers from syringes to test tubes (and vice versa) make their implementation long and difficult through the preparation of single-use sterile kits, as well as the maintenance of such sterility complicated.

An apparatus for tissue fragmentation is known from document US2020/0072712. The apparatus includes a canister defining a chamber with an opening, a lid, a shaft coupled to blades configured to cut the tissue during rotation.

Furthermore, the known methods do not allow even an immediate measurement of the quantity of molecules of the above mentioned types, unless after subsequent laboratory testing, leading to wide uncertainty about the effectiveness of the adopted methods.

### DISCLOSURE OF THE INVENTION

Purpose of the present invention is therefore to prevent the here mentioned inconveniences.

The invention, as characterized by the claims, achieves the purpose thanks to a breakthrough optimisation of the breaking up action of the processed biological material and to the resulting easy separation and extraction of mesenchymal cells and of the above mentioned molecules by means of single-use kits.

The main benefit of the present invention is essentially to reduce enormously the processing time of the biological material, from hours to a few minutes and therefore making it compatible even with the time of a surgical operation, preserving anyway the absolute sterility of the whole process, through the use of single-use kits, since it avoids the transfer of the obtained biological material from a container to another, from the beginning to the end of its processing.

The method used advantageously allows to avoid both centrifugation and labeling of the cells to be separated and does not foresee stressful actions for cells that may compromise their vitality.

The invention also allows a standardization of the method for processing biological material, wherein the measurement of the molecules quantity (called aliquotation) of the different types allows to determine the quality of the applied method and to know the composition of the product to be reinjected.

Finally, the product thus obtained is much more rich in growth factors and anti-inflammatories, although it has been obtained without the use of enzymes.

### BRIEF DESCRIPTION OF DRAWINGS

Further advantages and characteristics of the present invention will be more evident in the following detailed description, made with reference to the attached drawings, which represent a non-limiting example of execution thereof, wherein:

FIG. 1 illustrates the invention according to an overview in perspective;

FIG. 2 illustrates a detail of the invention according to a plan view from above, with an enlarged portion;

FIG. 3 illustrates a detail of the invention according to a view in perspective;

FIG. 4 illustrates the detail of FIG. 3 according to a longitudinal section;

FIG. 5 illustrates a portion of the invention according to an embodiment thereof;

FIG. 6 illustrates a detail of the invention according to the embodiment of FIG. 5 in longitudinal section.

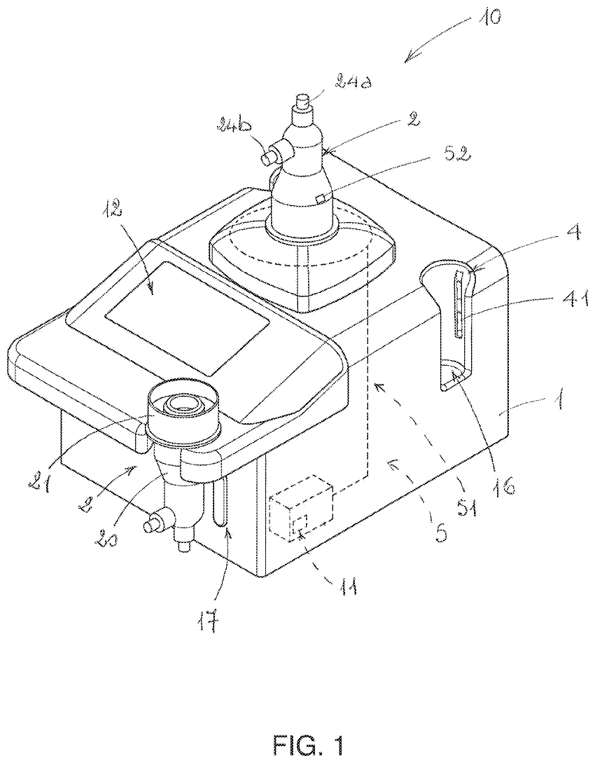

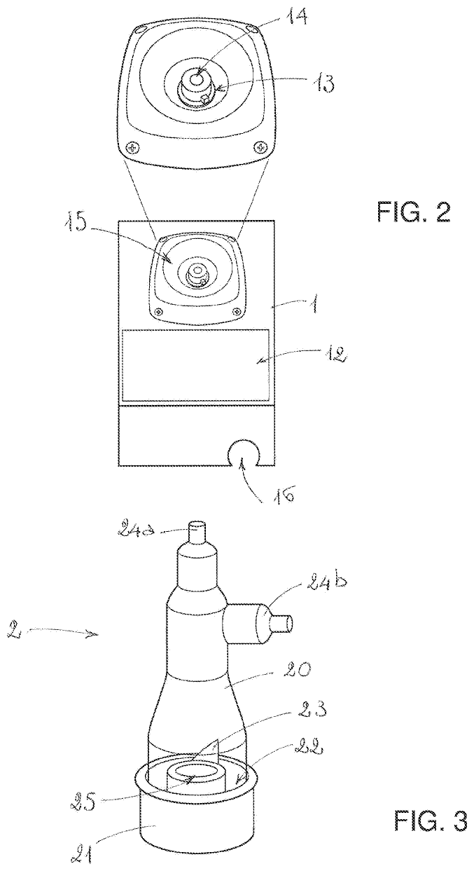

### PREFERRED EMBODIMENTS OF THE INVENTION

As shown in the figures, the invention concerns an apparatus for processing biological material, comprising a supporting structure (1), provided with a seat (15) and with a rotation device (13) supplied with constraining means (14), and a container (2) for biological material, provided with a shaped body (20), with a base (21), shaped to match the seat (15) of the supporting structure (1), and with at least one valve (24a) for extracting the biological material; the base (21) of the container (2) also supports movable cutting means (23), free to rotate relative to the base (21) and equipped with coupling means (25) for directly coupling to the constraining means (14) of the rotation device (13), in such a way as to be moved by this and acquire a relative motion relative to the container (2). The constraining means (14) of the rotation device (13) and the coupling means (25) of the movable cutting means (23) may be implemented in different ways functionally equivalent, such as, for example, pairs of magnets, pairs of complementary threads, or even bayonet joints or of other types.

The supporting structure (1), of such shape as to facilitate its external sterilisation, also comprises at least one housing (16) shaped to match the container (2), suitable for accommodating it at the end of the processing. When the container (2) is in said housings (16), an illuminator (17) is used for the transparent observation in such a way as to check how, in a few minutes, the disaggregated biological material, with previous addition of a water-based solution, is separated according to its density; measuring means (4) are then used for measuring the cellular fractions of the biological material inside the container (2). Said measuring means (4) comprise preferably at least one spectrophotometer (41), suitable for detecting the absorbance, that is the quantity of light absorbed by the biological material processed, in such a way as to define the concentration of the cellular fractions.

In a preferred embodiment, the base (21) and the body (20) of the container (2) comprise constraining means (20a, 21b) shaped to match each other, in such a way that they can be separated: also said constraining means (20a, 21b) can be implemented in different ways, like for example, complementary threads or bayonet joints. Moreover, the container (2) can be provided with a second valve (24b), which can be used both to introduce the biological material without opening the container (2) and to have an additional passage to extract the same at the end of the processing.

In addition to the movable cutting means (23), it is desirable to have inside the container (2) also fixed cutting means (22), suitable for promoting the fragmentation of the biological material.

The fixed cutting means (22) and/or the movable cutting means (23) preferably consist of blades.

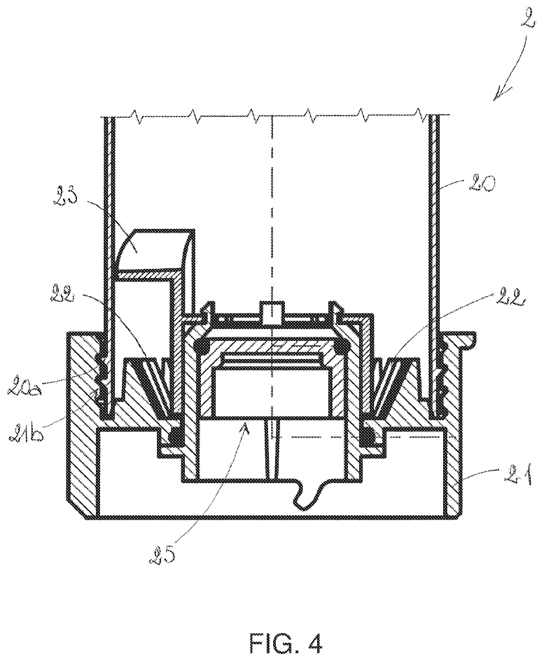

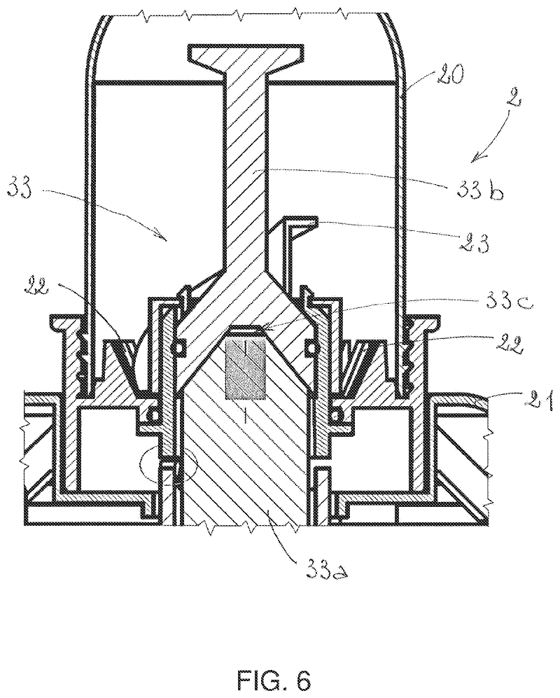

In the embodiment shown, in particular in FIGS. 4 and 6, the movable cutting means (23) which also have the function of mixing the biological material with the water-based solution, advantageously comprise a ring-shaped base and a protrusion which extends substantially from the base in the direction of the rotation axis. The protrusion has, at the free end, an extension which extends transversely relative to the protrusion. In an embodiment the protrusion and/or the extension comprises a blade.

In the embodiment shown, the fixed cutting means (22) are circumferentially distributed and arranged between the movable cutting means (23) and the base (21).

The breaking up process takes place through the cutting action performed by the blades rotating at high speed within the mass of biological material.

The fixed blades, placed near the bottom of the container (2), are inclined in such a way as to push the biological material against the walls of the container (2), causing a breaking up of the material both by cutting and by impact.

In an embodiment not shown, the circumferentially distributed cutting means (22) can be rotated and the cutting means (23) including the protrusion can be held still.

In an alternative embodiment, the container for processing biological material comprises a shaped body (20), a base (21) supporting first mixing or cutting means and second cutting means (22, 23), at least a valve (24a) for extracting the biological material; wherein the first mixing or cutting means and the second cutting means are equipped with coupling means (25) for coupling to a rotation device (13), so as to acquire both a relative motion relative to the container (2), wherein the first mixing or cutting means are free to rotate relative to the base (21) in a first direction and the second cutting means are free to rotate relative to the base 21 in a second direction opposite to the first direction.

In this embodiment, the first mixing or cutting means and the second cutting means are therefore relatively movable with respect to each other.

Inside the supporting structure (1) a dedicated software (11) is installed, connected to a screen (12) for displaying the operating specifications. The software (11) allows also to use a recognition device (5) for recognising the containers (2), suitable for unambiguously identifying each container (2). The recognition device (5) for recognising the containers (2) comprises identification means (52), for example a barcode or a RFID chip, fixed to the container (2), and a reader (51), for example respectively an optical reader or an antenna, fixed to the supporting structure (1), wherein the reader (51) and the identification means (52) are able to communicate by means of the dedicated software (11) installed in the supporting structure (1). This allows the apparatus (10) to recognize each container (2), verify its expiry date, inhibit its reuse and decide its type of processing before giving the consent to operation. The software (11) also allows to communicate with remote stations in order to transmit and save data, measurements taken and operating specifications for each container (2) used.

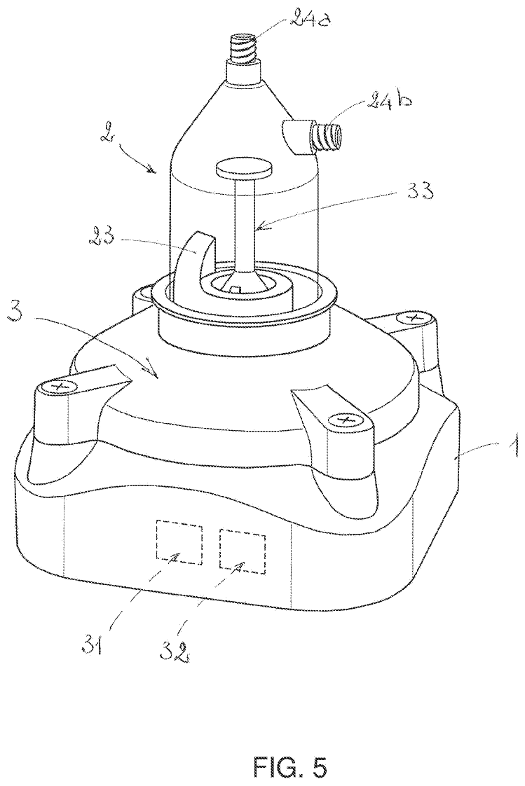

Finally, in a preferred embodiment, shown in FIG. 5, the apparatus (10) comprises an ultrasonic device (3), suitable for optimizing the disaggregation of the biological material. The ultrasonic device (3), to be used when the integrity of the cells does not need to be maintained, comprises an electric generator (31), a converter (32) for converting electrical energy into mechanical energy, suitable for generating ultrasonic waves, and at least one sonotrode (33), suitable for spreading the ultrasonic waves in the biological material in such a way as to promote its disaggregation. The ultrasonic device (3) allows in fact to generate waves with frequency so high that, intensified by the sonotrode (33), they cause the phenomenon of cavitation in the liquid in which they propagate, acting on the biological material as a blade at extremely high temperature which is capable of breaking the cell membrane, releasing the molecules which are inside the cells and removing the non-desirable cells.

The sonotrode (33) comprises a bottom (33a) fixed to the supporting structure (1), and an emitter (33b), fixed to the container (2): between the bottom (33a) and the emitter (33b) are interposed constraining means (33c) suitable for allowing their quick uncoupling, which preferably consist of magnets.

The method for processing biological material implemented by the described apparatus (10) thus foresees at least the steps of:
extraction of the biological material;
addition of a water-based solution to the extracted biological material;
mechanical agitation of the mixture of biological material and water-based solution;
settling, for the separation of the mixture in layers of different density;
extraction of the lower layer, rich in stem cells and anti-inflammatory and growth factors, called SVF (stromal vascular fraction).

The biological material processed can derive from different tissue areas, such as, for example, adipose tissue (obtained by liposuction or by other means), cord tissue, or other tissues.

The quantity of water-based solution (for example salt solution, physiological solution or even distilled water) is between ⅕ and 5 times the quantity of biological material, in volume.

The mechanical agitation preferably consists in a rotation of the mixture of biological material and water-based solution at a speed between 50 and 5000 revolutions per minute for a time between 5 seconds and 5 minutes.

The settling has a duration between 2 and 20 minutes, suitable to be reconciled with surgical times.

In summary, the apparatus (10) allows in particular the processing of tissue obtained by liposuction with closed-circuit, using a single-use sterile container (2), to obtain microfragmented adipose tissue with minimal manipulation intended for an autologous graft (but not only) in a one-stage surgical procedure. The tissue thus processed is rapidly separable into phases by gravitation and allows to procure exclusively the phases of interest, that is to say those rich in factors (growth and anti-inflammatory) and in stem cells.

1. An apparatus for processing biological material, characterised in that it comprises:
a supporting structure, provided with a seat and with a rotation device supplied with constraining means;
a container for biological material, provided with a shaped body, with a base, shaped to match the seat of the supporting structure and supporting movable cutting means free to rotate relative to the base, and fixed cutting means, suitable for promoting the fragmentation of the biological material; and with at least one valve for extracting the biological material; said movable cutting means being equipped with coupling means for directly coupling to the constraining means of the rotation device in such a way as to acquire a relative motion relative to the container.

2. The apparatus according to claim 1, wherein the supporting structure comprises a dedicated software and a screen for displaying the operating specifications.

3. The apparatus according to claim 1, wherein the container comprises at least one second valve for extracting and/or introducing biological material.

4. The apparatus according to claim 1, wherein the base and the body of the container comprise constraining means shaped to match each other, in such a way that they can be separated.

5. A container for processing biological material, comprising a shaped body, wherein it comprises:
a base, supporting movable cutting means free to rotate relative to the base;
fixed cutting means, suitable for promoting the fragmentation of the biological material;
at least one valve for extracting the biological material;
said movable cutting means being equipped with coupling means for coupling to a rotation device, in such a way as to acquire a relative motion relative to the container.

6. The container according to claim 5, wherein the container comprises at least one second valve for extracting and/or introducing biological material.

7. The container according to claim 5, wherein the base and the body of the container comprise constraining means shaped to match each other, in such a way that they can be separated.

8. The apparatus according to claim 1, wherein it comprises an ultrasonic device, suitable for breaking up the biological material.

9. The apparatus according to claim 8, wherein the ultrasonic device comprises an electric generator, a converter for converting electrical energy into mechanical energy, suitable for generating ultrasonic waves, at least one sonotrode, suitable for spreading the ultrasonic waves in the biological material in such a way as to promote its disaggregation.

10. The apparatus according to claim 9, wherein the sonotrode comprises a bottom fixed to the supporting structure, and an emitters, fixed to the container, interposed between the bottom and the emitter there being constraining means suitable for allowing their quick uncoupling.

11. The apparatus according to claim 10, wherein the constraining means comprise magnets.

12. The apparatus according to claim 1, wherein the supporting structure comprises at least one housing shaped to match the container, suitable for accommodating it at the end of the processing.

13. The apparatus according to claim 1, wherein it comprises measuring means for measuring the cellular fractions of the biological material inside the container.

14. The apparatus according to claim 13, wherein the measuring means comprise at least one spectrophotometer, suitable for detecting the absorbance of the biological material processed for defining the concentration of the cellular fractions.

15. The apparatus according to claim 2, wherein it comprises a recognition device for recognising the containers, suitable for unambiguously identifying each container.

16. The apparatus according to claim 15, wherein the recognition device for recognising the containers comprises identification means fixed to the container, a reader fixed to the supporting structure, said reader and said identification means being able to communicate by means of the dedicated software installed in the supporting structure.

17. The apparatus according to claim 1, wherein the supporting structure comprises a transparency illuminator for checking the separation into phases of the biological material.

18. A method for processing biological material, wherein it comprises at least the steps of:
extraction of the biological material;
addition of a water-based solution to the extracted biological material;
mechanical agitation of the mixture of biological material and water-based solution;
settling, for the separation of the mixture in layers of different density;
extraction of the lower layer, rich in stem cells and anti-inflammatory and growth factors, called SVF (stromal vascular fraction).

19. The method according to claim 18, wherein the quantity of water-based solution is between ⅕ and 5 times the quantity of biological material in volume.

20. The method according to claim 18, wherein the mechanical agitation consists in a rotation of the mixture of biological material and water-based solution at a speed between 50 and 5000 revolutions per minute for a time between 5 seconds and 5 minutes.

21. The method according to claim 18, wherein the settling has a duration ranging between 2 and 20 minutes.
